(19) United States
(12) Patent Application Publication
(10) Pub. No.: US 2024/0050319 A1
(43) Pub. Date: Feb. 15, 2024

(54) MEDICAL DEVICE WITH INTEGRATED INSTRUMENT AND RELATED METHODS

(71) Applicant: Boston Scientific Scimed, Inc., Maple Grove, MN (US)

(72) Inventors: Bryan BANNON, Duxbury, MA (US); James WELDON, Newton, MA (US); Jeff INSULL, Northborough, MA (US)

(73) Assignee: Boston Scientific Scimed, Inc., Maple Grove, MN (US)

(21) Appl. No.: 18/447,608

(22) Filed: Aug. 10, 2023

Related U.S. Application Data
(60) Provisional application No. 63/371,089, filed on Aug. 11, 2022.

(51) Int. Cl. A61J 15/00; A61B 1/06; A61B 1/00; A61B 1/015

(52) U.S. Cl. CPC A61J 15/0023; A61J 15/0019; A61B 1/06; A61B 1/00066; A61B 1/015

(57) ABSTRACT

A medical device includes a tube comprising a lumen extending from a proximal end to a distal end of the tube, a cap at the distal end of the tube having a distal opening corresponding to the lumen, and an imaging system positioned on a distal-facing surface of the cap. The medical device also includes an instrument at least partially disposed in the lumen that is non-removable from the medical device, an instrument actuation mechanism for controlling the instrument, and a single handle connected to the proximal end of the tube and housing the instrument actuation mechanism. The instrument actuation mechanism, to which a proximal end of the instrument is affixed, may be configured to be moved between a first and second position on the single handle to control an extension and retraction of the instrument relative to the tube.

### CROSS-REFERENCE TO RELATED APPLICATIONS

This application claims the benefit of priority under 35 U.S.C. § 119 from U.S. Provisional Application No. 63/371,089, filed Aug. 11, 2022, which is incorporated by reference herein in its entirety.

### TECHNICAL FIELD

The disclosure relates generally to medical devices. More specifically, aspects of the disclosure pertain to medical devices having an integrated instrument and methods of using such medical devices.

### BACKGROUND

Traditionally, a percutaneous tracheostomy to place an endotracheal tube and a percutaneous endoscopic gastrostomy (PEG) to place a feeding tube are separate procedures. These separate procedures are visually guided by different types of endoscopic medical devices, and are performed by different specialists at different sites of service. For example, the percutaneous tracheostomy may be performed by a surgeon, a critical care pulmonologist, and/or an interventional pulmonologist bedside or in a bronchoscopy suite using a bronchoscope. On the other hand, the PEG may be performed by a surgeon or gastroenterologist in an operating room or gastrointestinal suite using a gastroscope.

More recently, a specialist may be trained to consecutively perform both the percutaneous tracheostomy and the PEG bedside for patients that are in the intensive care unit or other similar setting using a same endoscopic medical device, such as a bronchoscope. These bedside settings are limited in space, particularly near the patient's head, where the specialist operating the bronchoscope is positioned. Moreover, one or more steps of the PEG may rely on an assistant (e.g., a second operator), for example, to facilitate insertion of a separate accessory device (e.g., a snare) into the bronchoscope and/or operation of the separate accessory device. However, due to the limited space in bedside settings, the insertion and/or use of the separate accessory device (e.g., the snare) may be cumbersome, for example, prolonging the procedure, increasing risks during the procedure, etc.

### SUMMARY

In one example, a medical device may comprise a tube having a proximal end and a distal end that includes a lumen extending from the proximal end to the distal end, a cap at the distal end of the tube having a distal opening corresponding to the lumen, and an imaging system positioned on a distal-facing surface of the cap. The medical device may also comprise an instrument at least partially disposed in the lumen that is non-removable from the medical device, an instrument actuation mechanism for controlling the instrument, and a single handle connected to the proximal end of the tube and housing the instrument actuation mechanism. A proximal end of the instrument may be affixed to the instrument actuation mechanism and the instrument actuation mechanism may be configured to be moved between a first position and a second position on the single handle to control an extension and retraction of the instrument relative to the tube. A distal end of the instrument may be retracted relative to the tube in a collapsed state when the instrument actuation mechanism is in the first position. The distal end of the instrument may be extended relative to the tube in an expanded state when the instrument actuation mechanism is in the second position.

In any of the exemplary medical devices disclosed herein, the distal end of the tube may include an articulation joint, and the distal end of the instrument may be positioned within the articulation joint when the instrument actuation mechanism is in the first position. The single handle may further house an articulation actuation mechanism for controlling the articulation joint, where the articulation joint may be configured to bend in at least two directions away from a longitudinal axis of the tube.

Additionally, the instrument may be extended relative to the tube via the distal opening, and the distal opening may include a clamshell shape to lock an orientation of the instrument relative to the medical device as the instrument is extended. The medical device may further comprise a connector disposed within the single handle. The connector may include a plurality of connector ports, and a distal opening connecting the single handle to the proximal end of the tube. At least one of the plurality of connector ports may include an instrument connector port configured to receive the instrument from the instrument actuation mechanism and the instrument received via the instrument connector port may extend into the lumen of the tube via the distal opening.

In some aspects, the instrument actuation mechanism may include an instrument actuator configured to be moved between the first position and the second position, a link, and a driver connected to the instrument actuator via the link to which the proximal end of the instrument is attached.

In other aspects, the medical device may include a sheath at least partially disposed within the lumen. The instrument may be disposed within the sheath. The instrument actuation mechanism may include a sheath actuator that extends and retracts the sheath in which the instrument is disposed relative to the tube, and an instrument actuator that extends and retracts the instrument relative to the sheath to transition the instrument between the collapsed state and the expanded state.

In additional aspects, the instrument may be a wire. A first end of the wire may be attached to a fixation point on the distal-facing surface of the cap, and the wire may extend from the fixation point through a second distal opening of the cap, different from the distal opening corresponding to the lumen, into the handle via the tube. A second end of the wire opposite the first end of the wire may be affixed to the instrument actuation mechanism. When the instrument actuation mechanism is in the first position, a first portion of the wire from the fixation point to the second distal opening may form a loop in the collapsed state that at least partially contacts the distal-facing surface. When the instrument actuation mechanism is in the second position, a second portion of the wire may be extended from the tube via the second opening to transition the loop to the expanded state. The distal-facing surface of the cap may comprise a recess arranged between the fixation point and the second distal opening, where the recess may be configured to receive and secure an object captured by the instrument.

In further aspects, the instrument may be a wire. A first end of the wire may extend through a second distal opening of the cap, different from the distal opening corresponding to the lumen, into the handle via the tube to attach to the instrument actuation mechanism. A second end of the wire may extend through a third distal opening in the cap, different from the distal opening corresponding to the lumen and the second distal opening, into the handle via the tube to attach to the instrument actuation mechanism. When the instrument actuation mechanism is in the first position, a first portion of the wire from the second distal opening to the third distal opening may form a loop in the collapsed state that at least partially contacts the distal-facing surface. When the instrument actuation mechanism is in the second position, a second portion of the wire may be extended from the tube via the second opening and a third portion of the wire may be extended from the tube via the third opening to transition the loop to the expanded state.

Additionally, the single handle may further comprise a fluidics device having a proximal end connectable to one or more of a fluid supply source and a vacuum source and a distal end connected to the proximal end of the tube. The fluidics device may be configured to supply fluid and/or apply suction to a patient's body via the distal opening. The imaging system may include an imaging device positioned within an imaging port on the distal-facing surface of the cap, and an illumination device positioned within an illumination port on the distal-facing surface of the cap.

In another example, a medical device may comprise a tube having a proximal end and a distal end that includes a lumen extending from the proximal end to the distal end, a cap at the distal end of the tube having a distal opening corresponding to the lumen, a sheath at least partially disposed within the lumen, and an instrument disposed within the sheath, where the sheath and the instrument are non-removable from the medical device. The medical device may also comprise an instrument actuation mechanism comprising a sheath actuator and an instrument actuator, and a single handle connected to the proximal end of the tube and housing the instrument actuation mechanism. A proximal end of the sheath may be affixed to the sheath actuator, and the sheath actuator may be configured to move between a first position and a second position on the single handle to control an extension and retraction of the sheath in which the instrument is disposed relative to the tube via the distal opening. A proximal end of the instrument may be affixed to the instrument actuator, and the instrument actuator may be configured to move between a third position and a fourth position on the single handle to control an extension and retraction of a distal end of the instrument relative to the sheath to transition the distal end of the instrument between a collapsed state and an expanded state.

Any of the exemplary medical devices disclosed herein may include any of the following features. The distal end of the tube may include an articulation joint, and a distal end of the sheath, in which the distal end of the instrument is disposed, may be positioned within the articulation joint when the sheath actuator is in the first position. The instrument may be a snare and the medical device may be configured to be used for a PEG.

In a further example, a medical device may comprise a tube having a proximal end and a distal end that includes a lumen extending from the proximal end to the distal end, a cap at the distal end of the tube having a distal opening corresponding to the lumen, a handle connected to the proximal end of the tube and housing an actuation mechanism configured to be moved between a first position and a second position on the handle, and a wire having a first end and a second end, where the wire may be at least partially disposed in the lumen. The first end of the wire may be attached to a fixation point on a distal-facing surface of the cap and extend from the fixation point through a second distal opening of the cap, different from the distal opening corresponding to the lumen, into the handle via the tube. A second end of the wire opposite the first end of the wire may be affixed to the actuation mechanism. When the actuation mechanism is in the first position, a first portion of the wire from the fixation point to the second distal opening may form a loop in a collapsed state that at least partially contacts the distal-facing surface. When the actuation mechanism is in the second position, a second portion of the wire may be extended from the tube via the second opening to transition the loop to an expanded state.

Any of the exemplary medical devices disclosed herein may include any of the following features. The distal-facing surface of the cap may comprise a recess arranged between the fixation point and the second distal opening, the recess configured to receive at least a portion of an object captured by the wire.

It may be understood that both the foregoing general description and the following detailed description are exemplary and explanatory only and are not restrictive of the invention, as claimed. As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” The term “distal” refers to a direction away from an operator/toward a treatment site, and the term “proximal” refers to a direction toward an operator. The term “approximately,” or like terms (e.g., “substantially”), includes values +/−10% of a stated value.

### BRIEF DESCRIPTION OF THE DRAWINGS

The accompanying drawings, which are incorporated in and constitute a part of this specification, illustrate examples of this disclosure and together with the description, serve to explain the principles of the disclosure.

FIGS. 1A-1D depict various views of an exemplary medical device for performing a medical procedure.

FIGS. 2A-2D depict block diagrams of another exemplary medical device for performing a medical procedure.

FIGS. 3A-3C depict perspective views of a distal portion of a further exemplary medical device for performing a medical procedure in various configurations.

FIG. 4 depicts an exemplary process to perform a medical procedure using the exemplary medical device of FIGS. 1A-1D.

### DETAILED DESCRIPTION

As briefly mentioned above, specialists may now be trained to consecutively perform both a percutaneous tracheostomy and a PEG bedside for patients that are in the intensive care unit or other similar bedside settings using a same or single endoscopic medical device. For example, after the endotracheal tube is placed to complete the percutaneous tracheostomy, the medical device may be advanced into the stomach and operated by the specialist (e.g., a first operator) to insufflate and obtain visualization necessary for determining an access site. Once the access site is determined, a needle surrounded by an outer cannula may be inserted into the stomach, and the needle may be removed such that the cannula remains. A PEG wire may be inserted into the stomach via the cannula.

In the meantime, an assistant (e.g., a second operator) that is positioned near the first operator of the medical device may feed a distal portion of an accessory device separate from the medical device, such as a snare, into a working lumen of the medical device via an access port and through a distal opening at a distal end of the medical device. The snare may be delivered in a collapsed state, and may then be actuated, via a handle portion of the accessory device, by the second operator to cause the snare to expand. In the expanded state, the snare may be used to capture the PEG wire when the PEG wire is inserted into the stomach via the cannula. The second operator, in some cases, may also utilize controls on the handle portion of the accessory device to rotate the snare when in the expanded state to better position the snare relative to the PEG wire to be captured. However, having the second operator present to assist with the insertion and operation of the separate accessory device may be cumbersome in the limited space of an intensive care unit or other similar bedside setting, for example, prolonging the procedure, increasing risks during the procedure, etc.

Therefore, aspects of this disclosure are directed to a medical device having a non-removable, integrated instrument that enables a single operator to control the medical device as well as the integrated instrument, eliminating the need for a second operator to assist with the insertion and operation of the instrument during a medical procedure, such as a PEG. The medical device may include a single handle and a tube extending distally from the handle. The single handle may be sized to be held by an operator and configured to house various components of the medical device within and/or on the handle, including an instrument actuation mechanism for controlling the integrated instrument. At least a distal portion of the tube may be inserted into a patient during the medical procedure, and the tube may include at least one lumen in which the integrated instrument may be at least partially disposed. Moreover, the integrated instrument may be transitioned between collapsed and expanded states via the instrument actuation mechanism, for example, on and/or integrated into the handle.

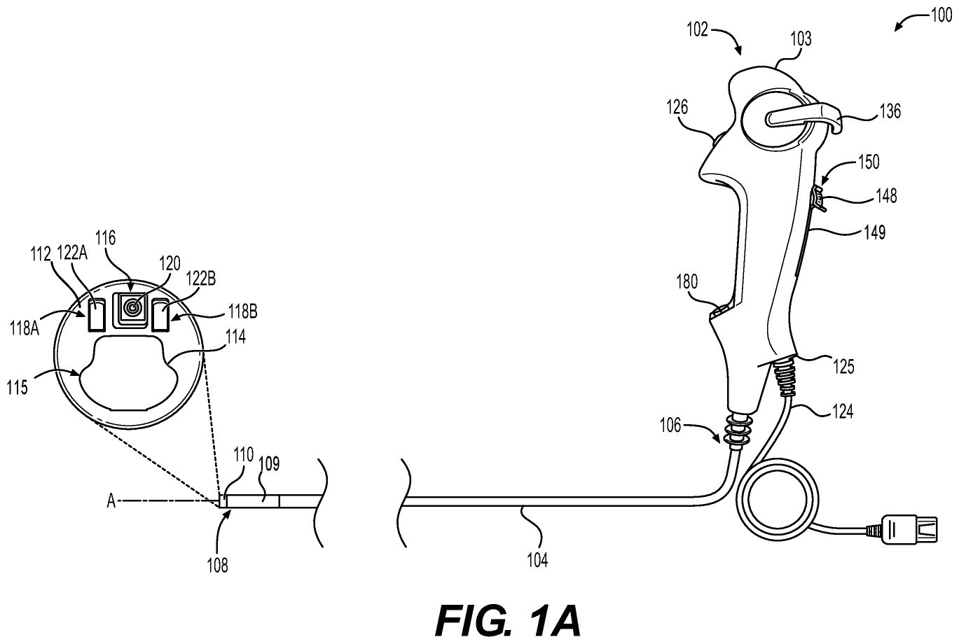

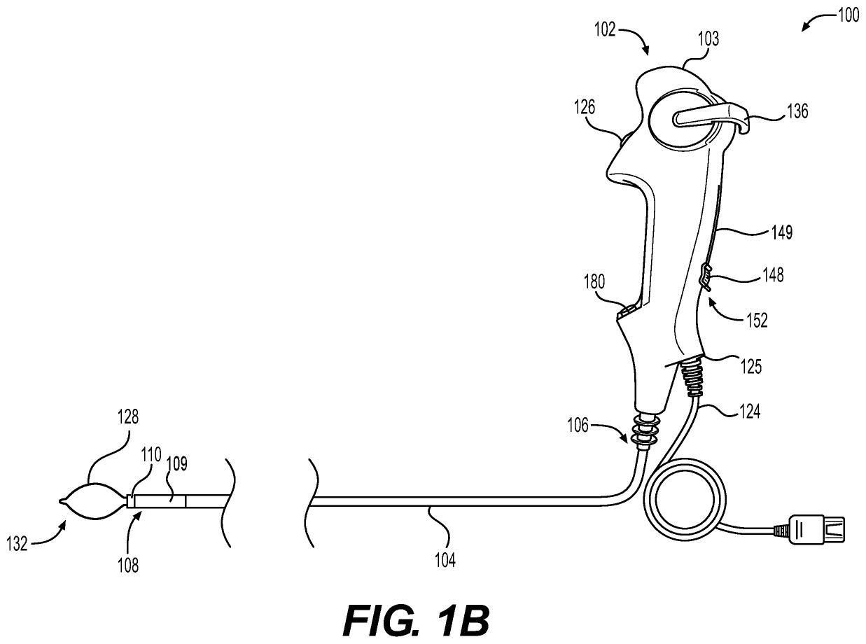

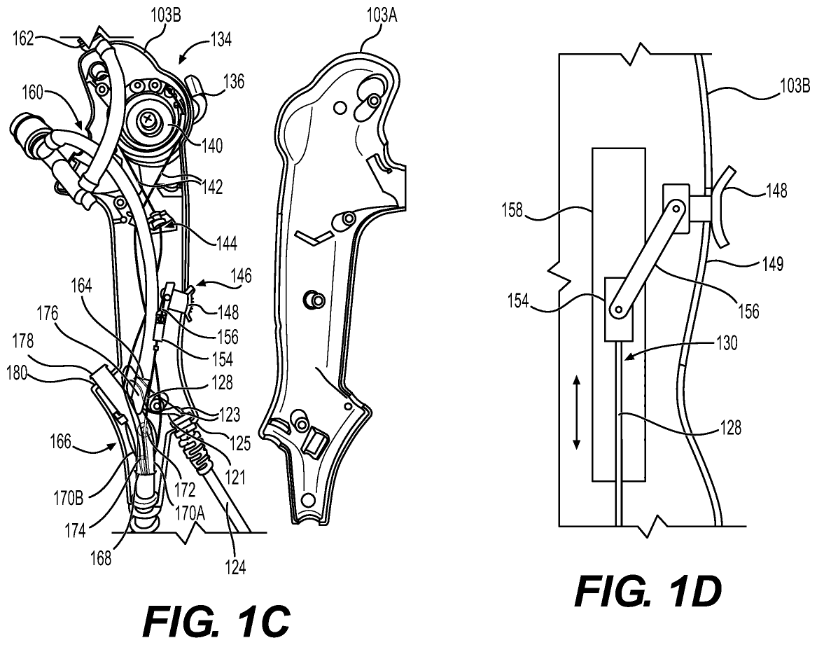

The instrument may be integrated into the medical device in various ways. In one example and as described with reference to FIGS. 1A-1C below, the lumen of the tube may serve as a sheath for the instrument. In this example, a distal end of the instrument may be extended and retracted relative to the tube to transition between the collapsed and expanded states when the instrument actuation mechanism is actuated by the single operator of the medical device.

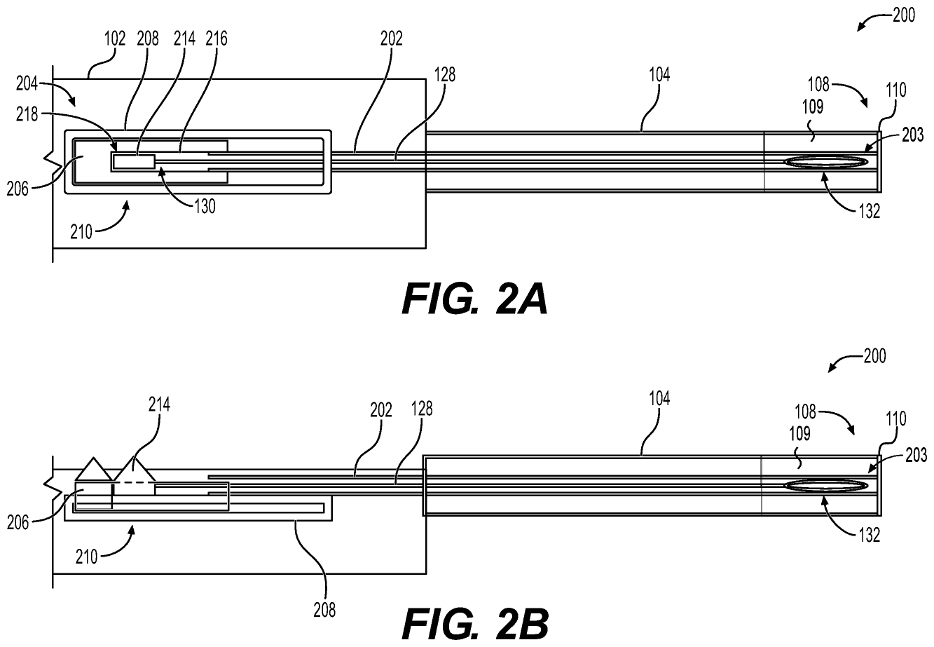

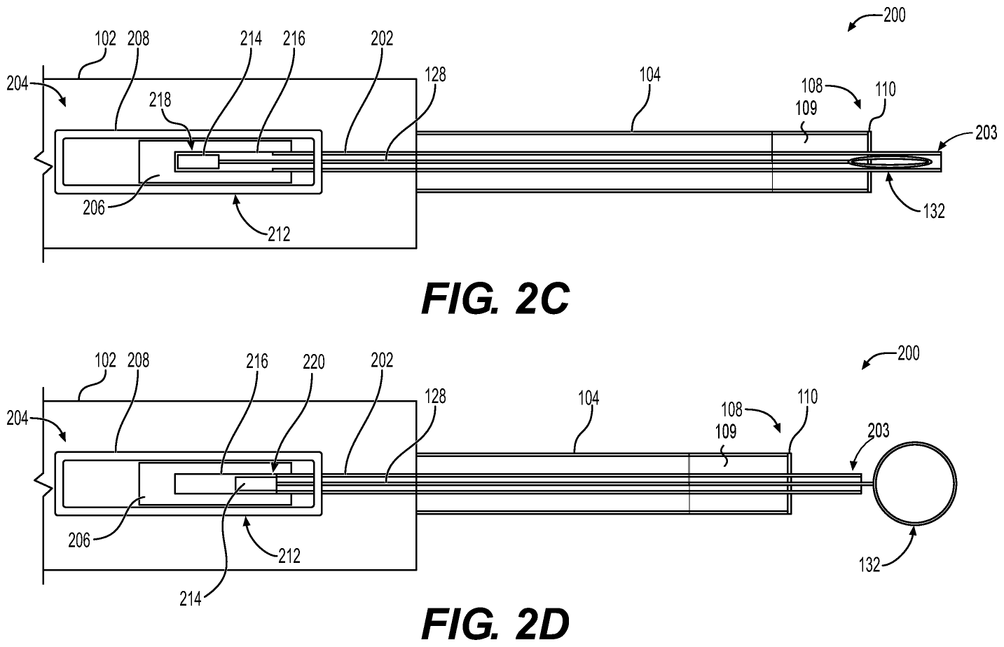

In another example and as described with reference to FIGS. 2A-2D below, the instrument may be disposed in a sheath separate from the lumen. The sheath with the instrument disposed therein may be extended and retracted relative to the tube when a first actuator of the instrument actuation mechanism is actuated by the single operator of the medical device. The instrument may then be extended and retracted relative to the sheath to transition between the collapsed and expanded states when a second actuator of the instrument actuation mechanism is actuated by the single operator of the medical device.

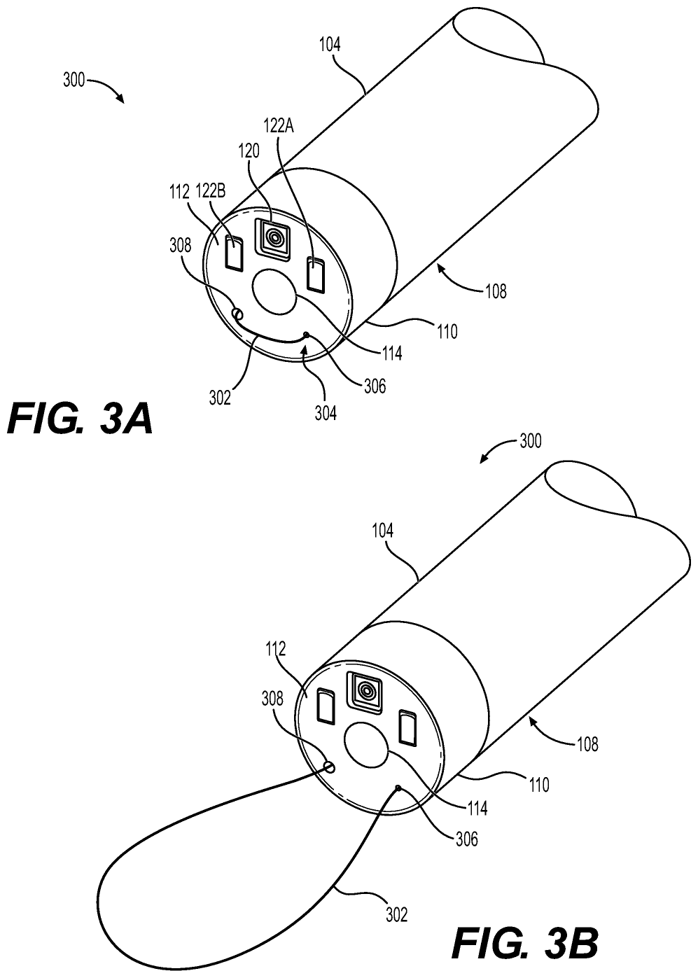

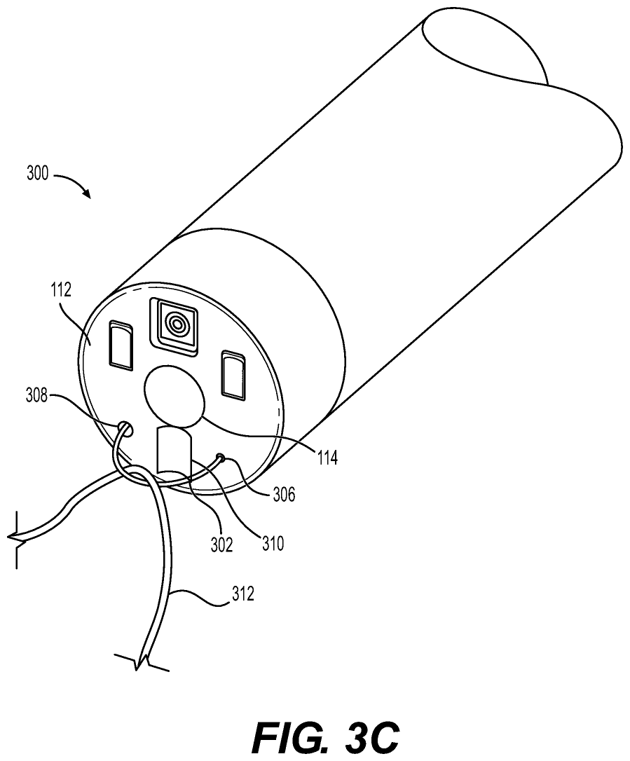

In a further example and as described with reference to FIGS. 3A-3C below, the instrument may be a wire. In this further example, a first end of the wire is attached at a fixation point to a distal-facing surface of a cap at the distal end of the tube and a second end of the wire extends proximally into the cap (e.g., via a distal opening in the cap) and through the lumen of the tube to attach to an instrument actuation mechanism within the handle. In a collapsed state, at least a portion of the wire from the fixed point to the point of entry into the cap may contact the distal-facing surface of the cap. When the instrument actuation mechanism is actuated by the single operator of the medical device, the wire may be extended and retracted via the distal opening in the cap to transition between the collapsed and expanded states. Alternatively, rather than the first end of the wire being attached to a fixation point, the cap may include another distal opening, and similar to the second end of the wire, the first end of the wire may extend into the cap (e.g., via the other distal opening in the cap) and through the lumen of the tube to attach to the same or a different instrument actuation mechanism within the handle to provide two-sided actuation.

FIGS. 1A-1D depict an exemplary medical device 100 for performing a medical procedure. One example medical procedure may include a PEG performed subsequent to a percutaneous tracheostomy, where both procedures may be performed using medical device 100. FIG. 1A depicts medical device 100 in a first configuration where an integrated instrument (not shown in FIG. 1A) of medical device 100 is retracted and in a collapsed state. Medical device 100 may be an endoscope or other type of scope, such as a bronchoscope, ureteroscope, duodenoscope, gastroscope, endoscopic ultrasonography (“EUS”) scope, colonoscope, laparoscope, arthroscope, cystoscope, aspiration scope, sheath, or catheter. Medical device 100 may include a handle 102 and a tube 104 (i.e., a shaft) extending distally from handle 102. The integrated instrument may be a device or tool, such as a snare, a net, a basket, a forceps, a grasper, scissors, a clip, a stapler, a needle, a knife, an electrode, a cautery loop, or other similar type of instrument for acting upon an object of interest during the medical procedure, among other examples. The instrument may be specific to the medical procedure being performed. For example, for the PEG, the instrument may be a snare that is used to capture a PEG wire, as discussed in more detail below. The instrument may be integrated with medical device 100 such that the instrument is non-removable from medical device 100 and can be controlled via handle 102 by a single operator of medical device 100 to extend and retract the integrated instrument, which may transition the integrated instrument between collapsed and expanded states.

Handle 102 may include a housing 103 sized and/or shaped to be held by a single hand of the operator of medical device 100, and various components of medical device 100 may be disposed within and/or on housing 103, as described in detail below. A proximal end 106 of tube 104 may be attached to and extend distally from handle 102, where at least a distal portion of tube 104 may be inserted into a patient's body during the medical procedure.

In some examples, a cap 110 may be removably attached to a distal end 108 of tube 104. In other examples, cap 110 may be integrated with distal end 108 of tube 104. Although the term “cap” is used herein, it will be appreciated that the disclosure encompasses examples of medical devices 100 including any type of distal tip, including distal tips that are monolithically formed with tube 104 or otherwise formed without a cap. Tube 104 may include at least one lumen (not shown) for delivering one or more devices and/or wires, including the integrated instrument, from proximal end 106 to distal end 108 of tube 104. In some examples, the at least one lumen may be a working lumen. In other examples, tube 104 may include more than one lumen (e.g., at least one other lumen in addition to the working lumen).

A distal-facing surface 112 of cap 110 may include a distal opening 114 corresponding to the at least one lumen of tube 104 to enable one or more devices, including at least the integrated instrument, to extend distally from tube 104 to an intended site within the patient's body via distal opening 114. In some examples, and as shown in FIG. 1A, distal opening 114 may be sized and/or shaped (e.g., as a clam shell) to increase a surface area of distal opening 114 for improved fluidics performance of the medical device 100. The clam shell design may also enable the cap 110 to lock in an orientation of the integrated instrument relative to medical device 100 for more precise and predictable rotation when the integrated instrument is extended relative to tube 104 and in an expanded state, as discussed in greater detail below. In other examples, distal opening 114 may have a substantially circular configuration (e.g., may have a circular configuration, an elliptical configuration, or other similar configuration), as shown in FIGS. 3A-3C.

Medical device 100 may also include an imaging system at distal end 108 of tube 104. The imaging system may include an imaging device 120 and one or more illumination devices 122A, 122B (collectively illumination device(s) 122). Accordingly, distal-facing surface 112 of cap 110 may include an imaging port 116 to receive imaging device 120 and one or more illumination ports 118A, 118B (collectively illumination ports 118) to receive illumination device(s) 122. In some examples, the imaging system may be communicatively connected and controlled by a control unit (not shown) when medical device 100 is connected to the control unit via an external cable 124 (e.g., an umbilicus). As described in more detail below with reference to FIG. 1C, wires attached to imaging device 120 and illumination device(s) 122 may extend through tube 104 and into handle 102 to couple to or be encapsulated/contained within external cable 124.

Imaging device 120 may include a camera, an imaging sensor (such as, for example, a complimentary metal-oxide semiconductor or a CMOS sensor), a light sensor, or another image receiving device (such as, for example, a fiber optic imaging device). As described in more detail below with reference to FIG. 1C, imaging device 120 may capture image signals and transmit the image signals from medical device 100 to the control unit for processing. The control unit may provide the processed image signals for display as still and/or transient images via a display device of or associated with the control unit to provide the operator of the medical device 100 visual guidance during the medical procedure. Imaging device 120 may capture the image signals in response to control signals received from the control unit. Additionally, medical device 100 may include an image capture control element 126 disposed on handle 102 (e.g., on proximal portion of handle 102), and imaging device 120 may capture the image signals in response to an actuation of image capture control element 126. In some instances, actuation of image capture control element 126 may cause the captured image signals to be saved and/or recorded.

Illumination device(s) 122 may include any device configured to illuminate a target area within the patient's body (e.g., illuminate a site for the medical procedure) to facilitate imaging, among other aspects of the procedure (e.g., palpation for PEG). Illumination device(s) 122 may include, among others, bulbs, light emitting diodes (LEDs), one or more fiber optic cables, and light guides. While two illumination device(s) 122 are shown, in other examples, medical device 100 may only have one illumination device or may have more than two illumination devices. As described in more detail below with reference to FIG. 1C, illumination device(s) 122 may be controlled and/or operated based on control signals received from the control unit when medical device 100 is connected to the control unit via external cable 124. In some examples, the illumination device(s) 122 may direct light from an external light source. For example, the external light source may include one or more LED, Xenon, or other light sources of an illumination control system (not shown) that is coupled to the control unit.

Distal end 108 of tube 104 may also include an articulation joint 109 that enables distal end 108 and cap 110 to bend in at least two directions away from a longitudinal axis A of tube 104. In some examples, articulation joint 109 may extend to cap 110. The operator of medical device 100 may cause articulation joint 109 to bend in one of the at least two directions by actuating an articulation actuator 136 (e.g., on proximal portion of handle 102) in a given direction. Articulation actuator 136 may be one component of an articulation actuation mechanism of medical device 100 that is described in more detail below with reference to FIG. 1C. In some examples, when medical device 100 is in the first configuration where the integrated instrument is retracted relative to tube 104 and is in a collapsed state, as shown in FIG. 1A, a distal end of the integrated instrument may be positioned within the lumen at distal end 108 of the tube 104 (e.g., within articulation joint 109). This positioning of the distal end of the integrated instrument within articulation joint 109 may increase the rigidity, which may improve the pushability of articulation joint 109 when advancing through the patient's body. In this aspect, although positioning the integrated instrument within articulation joint 109 may decrease the flexibility of articulation joint 109, articulation joint 109 remains flexible enough not to significantly impact articulation, particularly for procedures such as PEG where a full articulation of 180 degrees is not necessary.

In addition to controlling the articulation of medical device 100, the operator may control the extension and retraction of the integrated instrument. For example, medical device 100 may include an instrument actuator 148, which may be positioned on handle 102. The operator of medical device 100 may cause medical device 100 to shift from the first configuration shown in FIG. 1A to a second configuration, as shown in FIG. 1B, by moving instrument actuator 148 from a first position 150 (FIG. 1A) to a second position 152 (FIG. 1B). In some aspects, instrument actuator 148 may be one component of an instrument actuation mechanism of medical device 100 that is described in more detail below with reference to FIG. 1C. In the second configuration of medical device 100 shown in FIG. 1B, a distal end 132 of integrated instrument 128 may be extended relative to tube 104, for example, out of distal opening 114 (FIG. 1A). Extending distal end 132 of integrated instrument 128 from distal opening 114 transitions distal end 132 from a collapsed state to an expanded state. In the expanded state, integrated instrument 128 may act upon (e.g., capture) one or more objects. In some examples, the instrument actuation mechanism may include a spring or biasing mechanism that facilitates a return of integrated instrument 128 from second position 152 to first position 150. Additionally or alternatively, the instrument actuation mechanism may include a locking feature that enables instrument actuator 148 to be locked at second position 152 to prevent integrated instrument 128 from unintentionally being retracted relative to tube 104 and transitioning at least partially back to a collapsed state.

Accordingly, single handle 102 of medical device 100 may include both the articulation actuation mechanism (e.g., articulation actuator 136) for controlling the bend of articulation joint 109 and the instrument actuation mechanism (e.g., instrument actuator 148) for controlling the extension and retraction of integrated instrument 128 to enable the single operator of medical device 100 to control both articulation joint 109 and integrated instrument 128. As most clearly shown in FIG. 1C, in addition to articulation actuator 136 and instrument actuator 148, each of a respective articulation actuation mechanism 134 and an instrument actuation mechanism 146 include components disposed within and/or on housing 103 of handle 102.

As shown in FIG. 1C, housing 103 may include at least a first housing portion 103A and a second housing portion 103B. First housing portion 103A and second housing portion 103B may be attachable to one another to form housing 103. FIG. 1C depicts a view of an interior of handle 102 of medical device 100 when first housing portion 103A is removed and medical device 100 is in the second configuration, as described with respect to FIG. 1B.

In addition to articulation actuation mechanism 134 and instrument actuation mechanism 146 described above, a fluidics device 160 and a connector 166 may also be disposed within and/or on housing 103 of handle 102. Connector 166 may be coupled to proximal end 106 of tube 104 via a distal opening 168 of connector 166. Connector 166 may be configured to connect components of handle 102 to tube 104. For example, connector 166 may include a plurality of connector ports for delivering instruments, tools, or wires, including integrated instrument 128, from handle 102 to tube 104, and/or applying fluid (e.g., gas) via the tube 104 to insufflate and/or suction, for example. Exemplary connector ports are discussed in turn below to describe how each component from handle 102 is connected via connector 166 to tube 104.

As previously discussed, articulation actuation mechanism 134 may include articulation actuator 136. At least a portion of articulation actuator 136 may be disposed on an exterior of handle 102 to enable the operator to interact with articulation actuator 136. For example, articulation actuator 136 may be gradually movable in a first direction (e.g., pushed upward in a proximal direction) by the operator to gradually bend distal end 108 of tube 104, including cap 110, in a first direction away from longitudinal axis A. Articulation actuator 136 may be gradually movable in a second direction opposite the first direction (e.g., pushed downward in a distal direction) by the operator to gradually bend distal end 108 of tube 104, including cap 110, in a second direction away from longitudinal axis A that is opposite from the first direction away from longitudinal axis A. Remaining components of articulation actuation mechanism 134 may be positioned within an interior of handle 102 and at least a portion of one or more of those components may extend to distal end 108 of tube 104. For example, an interior portion of articulation actuator 136 may be connected to an articulation wheel 140. An articulation wire 142 may be disposed around a circumference of articulation wheel 140. A first end of articulation wire 142 may extend through a first articulation connector port 170A of connector 166 into proximal end 106 of tube 104 via distal opening 168 of connector 166, and attach to a first side of articulation joint 109 at distal end 108 of tube 104. A second end of articulation wire 142 may extend through a second articulation connector port 170B of the connector, into proximal end 106 of tube 104 via distal opening 168 of connector 166, and attach to a second side of articulation joint 109 opposite the first side to which the first end of articulation wire 142 is attached) at distal end 108 of tube 104. In some examples, one or more sets of guide elements 144 positioned distally from articulation wheel 140 and proximally from connector 166 may be included within the interior of handle 102 to receive articulation wire 142 in order to guide and/or retain articulation wire 142 (e.g., to prevent entanglement and/or interference with other interior components of handle 102). In further examples, first articulation connector port 170A and/or first articulation port 170B may include a seal to facilitate the movement of articulation wire 142 as the operator actuates articulation actuator 136.

When articulation actuator 136 is gradually moved or pushed upward in the first direction by the operator, articulation wheel 140 may rotate in the same first direction that articulation actuator 136 is moved, causing the first end of articulation wire 142 to be pulled proximally. Pulling the first end of articulation wire 142 proximally may cause the first side of articulation joint 109 to be pulled proximally and distal end 108 of tube 104, including the cap 110, to bend in the first direction away from longitudinal axis A. Conversely, when articulation actuator 136 is gradually moved or pushed downward in the second direction by the operator, articulation wheel 140 may rotate in the same second direction that articulation actuator 136 is moved, causing the second end of articulation wire 142 to be pulled proximally. Pulling the second end of articulation wire 142 proximally may cause the second side of articulation joint 109 to be pulled proximally and distal end 108 of tube 104, including cap 110, to bend in the second direction away from longitudinal axis A. While articulation actuator 136 has been described and is shown as a lever mechanism that can be moved or pushed upward or downward, additional or alterative mechanisms may be used such as, for example, a siding mechanism, a rotating mechanism, a pushing mechanism, or the like.

As previously discussed, instrument actuation mechanism 146 may include instrument actuator 148. At least a portion of the instrument actuator 148 may be disposed within an track 149 on exterior of housing 103 of handle 102 to enable the operator to interact with and move instrument actuator 148 along track 149 between first position 150 and second position 152 shown in FIG. 1A and FIG. 1B, respectively. In some examples and as shown, track 149 on the exterior of housing 103 may follow a contour of handle 102 in an area where first housing portion 103A and second housing portion 103B connect. In other examples, handle 102 (e.g., first housing portion 103A or second housing portion 103B) may include a custom slot in which instrument actuator 148 may be disposed.

Instrument actuation mechanism 146 may also include a driver 154 within an interior of housing 103, as shown in FIG. 1C and FIG. 1D. Driver 154 may be connected to an interior portion of instrument actuator 148 via a link 156. Link 156 may be a rigid link having a first pivot attached to driver 154 and a second pivot attached to the interior portion of instrument actuator 148. In some examples, and as shown in FIG. 1D, driver 154 may be disposed within and configured to move along an interior track 158 (e.g., defined within an interior surface of housing 103) as instrument actuator 148 is moved between first position 150 and second position 152. A proximal end 130 of integrated instrument 128 may be attached to driver 154. Integrated instrument 128 may extend from driver 154 into connector 166 via an instrument connector port 172 of connector 166 and into proximal end 106 of tube 104 via distal opening 168 of connector 166. Integrated instrument 128 may then extend from proximal end 106 to distal end 108 of tube 104 within the at least one lumen of tube 104. Based on a position of instrument actuator 148, integrated instrument 128 may be extended or retracted relative to tube 104. In some examples, instrument connector port 172 of connector 166 may include a seal to facilitate the extension and retraction of integrated instrument 128 as instrument actuator 148 is moved by the operator.

When medical device 100 is in the first configuration where instrument actuator 148 is in first position 150 (shown in FIG. 1A), distal end 132 of integrated instrument 128 may be retracted relative to tube 104. For example, driver 154 and proximal end 130 of integrated instrument 128 may be in a proximalmost position within handle 102 and distal end 132 of integrated instrument 128 may be in a collapsed state and positioned within articulation joint 109 at distal end 108 of tube 104. As instrument actuator 148 is moved by the operator between first position 150 and second position 152, driver 154 and integrated instrument 128 correspondingly move in a distal direction, causing distal end 132 of integrated instrument 128 to extend distally from tube 104 via distal opening 114 in cap 110 and transition to an expanded state, as shown in the second configuration of the medical device 100 in FIG. 1B. The expanded state of integrated instrument 128 may enable an object of interest to be acted upon (e.g., captured) during the medical procedure.

As previously discussed, in some examples, distal opening 114 may be sized and/or shaped to lock in an orientation of the integrated instrument 128 in the expanded state relative to medical device 100 to facilitate rotation of integrated instrument 128. For example, when integrated instrument 128 is a snare, a widest portion 115 of distal opening 114 may receive and secure wires of the snare (e.g., at least partially based on a radial strain of the snare wires) causing the rotation of medical device 100 to correspond to a rotation of the snare. Resultantly, if the operator desires to rotate the snare to achieve a better position for capturing an object of interest, such as a PEG wire during a PEG, the operator may simply rotate medical device 100 with their hand in the desired direction (e.g., rather than having to actuate a separate rotation control mechanism) and the snare will rotate correspondingly.

Once the object of interest is positioned relative to integrated instrument 128 to enable capture of the object of interest (e.g., once the object is within or at least partially surrounded by distal end 132 of integrated instrument 128 in the expanded state), instrument actuator 148 may then be moved from second position 152 toward first position 150 to at least partially transition integrated instrument 128 back to a collapsed state and at least partially retract integrated instrument 128 relative to tube 104. In some instances, integrated instrument 128 may not be able to return to a fully collapsed state and/or fully retract back into tube 104 via distal opening 114 based on a size and/or shape of the object of interest. In such instances, instrument actuator 148 may be moved from first position 150 toward the second position 152 until a point at which the object of interest contacts distal-facing surface 112 of cap 110. An entirety of medical device 100 may then be removed from the patient. In some instances, medical device 100 may be a single-use or disposable device, and thus may be disposed of once removed. While instrument actuator 148 has been described herein as a sliding mechanism that is movable between first position 150 and second position 152, additional or alterative mechanisms may be used such as, for example, a rotating mechanism, a pushing mechanism, a lever mechanism, or the like.

Throughout the medical procedure, the imaging system at distal end 108 of tube 104, including imaging device 120 and illumination device(s) 122, may be utilized to provide the operator visualization of the procedure, among other aspects. As previously described, imaging device 120 and illumination device(s) 122 may transmit and/or receive signals from the control unit (not shown) when medical device 100 is connected to the control unit via external cable 124. Signals may be transmitted and/or received by imaging device 120 via a first wire 121 or a cable (fiber optic or another type of cable) communicatively coupling imaging device 120 to the control unit. Signals may be transmitted and/or received by illumination device(s) 122 via second wire(s) 123 or cables (fiber optic or another type of cable) communicatively coupling imaging illumination device(s) 122 to the control unit. First wire 121 attached to imaging device 120 and second wire(s) 123 attached to illumination device(s) 122 may extend proximally through tube 104 into handle 102 via distal opening 168 of connector 166. First wire 121 and second wire(s) 123 may then exit handle 102 via an imaging system connector port 174 of connector 166 that feeds first wire 121 and second wire(s) 123 into external cable 124 via an imaging system access port 125 (FIGS. 1A,1B, and FIG. 1C) of housing 103. In some examples, imaging system connector port 174 may include a seal to facilitate the feeding of (e.g., by retaining) first wire 123 and second wire(s) 123 into external cable 124.

During the medical procedure, fluidics device 160 may also be utilized to insufflate and/or apply suction, among other examples. A proximal end 162 of fluidics device 160 may include an umbilicus (not shown) positioned on an exterior of housing 103 that is connectable to a fluid supply source (not shown), and a distal end 164 of fluidics device 160 may be connected to a fluidics connector port 176 of connector 166. Resultantly, fluid supplied from the fluid supply source may be directed through fluidics device 160, into connector 166 via fluidics connector port 176, and into tube 104 via distal opening 168 of connector 166, where the fluid may further travel through tube 104 to supply fluid to patient's body via distal opening 114. Additionally and/or alternatively, proximal end of the fluidics device 160 may be connectable to a vacuum source (not shown), for example, via an umbilicus, to apply suction and enable fluid from the patient's body to be suctioned into tube 104 via distal opening 114. As previously discussed, distal opening 114 may be sized and/or shaped (e.g., as a clam shell) to increase a surface area of distal opening 114 for improved fluidics performance of the medical device 100. For example, the increased surface area of the clam shell size and shape over a substantially circular design may allow supply of fluid and/or application of suction at a greater rate and/or pressure.

In some embodiments, medical device 100 may be configured to receive accessory devices, tools, and/or instruments that are separate from (e.g., not integrated within and/or controlled by) medical device 100. In such examples, an exterior of housing 103 may include a working lumen access 180 connected to a working lumen connector port 178 of connector 166 within the interior of housing 103. Working lumen connector port 178 may receive the accessory devices, tools, and/or instruments that are inserted into medical device 100 via working lumen access 180 for delivery into the working lumen of the tube 104 via distal opening 168 of connector 166. In some examples, the working lumen may be the same lumen in which integrated instrument 128 is disposed, fluid is supplied, and/or suction is applied. In other examples, tube 104 may include more than one working lumen, and working lumen access 180 may provide access to a first working lumen, with the integrated instrument 128 in a second working lumen. The fluid and/or suction may be delivered and/or applied through one or both of the first working lumen and the second working lumen. The accessory devices, tools, and/or instruments may be extended distally through the working lumen of tube 104 and to an intended site within the patient's body via distal opening 114. The accessory devices, tools, and/or instruments may then be operated using controls of the respective accessory devices, tools, and/or instruments, and may be removed from medical device 100 once an associated task or step of the medical procedure is completed. In some embodiments, when integrated instrument 128 is the only instrument necessary or intended for the medical procedure, medical device 100 may not include working lumen access 180, as such access is not needed, and connector 166 may not include working lumen connector port 178.

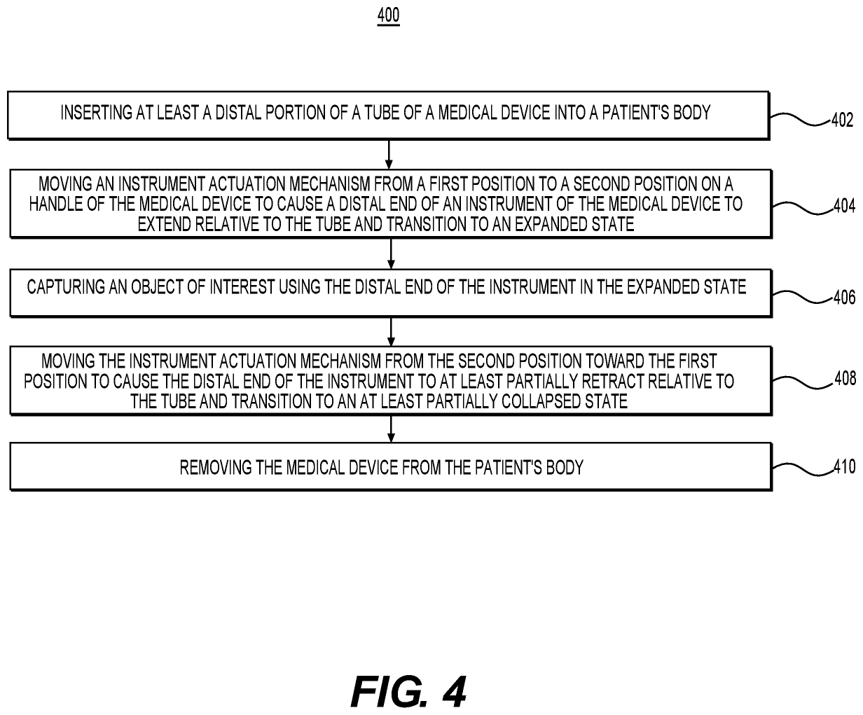

Referring now to FIG. 4, an exemplary process 400 to perform a medical procedure using medical device 100 of FIGS. 1A-1D may include steps 402-410. The medical procedure performed using medical device 100 may include an exemplary PEG procedure following a percutaneous tracheostomy. At step 402, process 400 includes inserting at least a distal portion of tube 104 of medical device 100 into a patient's body. To perform the PEG procedure following the percutaneous tracheostomy, distal portion (including distal end 108) of tube 104 may be advanced to a patient's stomach by an operator of medical device 100. The operator may be positioned near patient's head. In some aspects, an insufflation technique may be performed, where the fluid supply source may be connected to proximal end 162 of fluidics device 160 (e.g., the umbilicus) to cause gas to be forced through fluidics device 160, into tube 104 via connector 166, and into the patient's stomach via distal opening 114. Insufflation may cause the gastric wall to be brought in apposition with abdominal wall. Medical device 100 may be connected to the control unit via external cable 124, and tube 104 may be positioned within the stomach such that illumination device(s) 122 of the imaging system may emit light toward the abdominal wall to allow transillumination of the light through the abdominal wall that is perceivable external to the body.

An operator for placing the PEG tube, also referred to as a PEG operator, positioned near an abdomen of the patient (e.g., typically a different operator than the operator of medical device 100) may palpate the abdominal wall. The palpation may be captured by imaging device 120 of the imaging system, and corresponding images processed by the control unit may be displayed on the display device of or associated with the control unit to allow visualization and confirmation a puncture site. The PEG operator may insert a needle with an outer cannula into the puncture site, for example, using visualization from the imaging system (e.g., imaging device 120 and illumination device(s) 122) of medical device 100. The needle may be removed, and a PEG wire may be inserted via the cannula by the PEG operator.

At step 404, process 400 includes moving an instrument actuation mechanism (e.g., instrument actuator 148) from a first position (i.e., first position 150) to a second position (i.e., second position 152) to cause distal end 132 of integrated instrument 128 to extend relative to tube 104 and transition to an expanded state. For example, prior to or while the PEG wire is being inserted, the operator of medical device 100 may move instrument actuator 148 from first position 150 to second position 152 such that driver 154 correspondingly moves integrated instrument 128 (e.g., an integrated snare) to extend distal end 132 of integrated instrument 128 distally relative to tube 104. Extending distal end 132 of integrated instrument 128 relative to tube 104 may transition distal end 132 to an expanded state for capturing the PEG wire, for example, as it is inserted into the stomach via the cannula. The operator of medical device 100 may rotate medical device 100 (e.g., handle 102 and tube 104) as needed to correspondingly rotate integrated instrument 128 in order to better position distal end 132 of integrated instrument 128 relative to the PEG wire.

At step 406, process 400 includes capturing an object of interest (e.g. the PEG wire) using distal end 132 of integrated instrument 128 in the expanded state. In some examples, the operator of medical device 100 may rotate, extend or retract, or otherwise manipulate integrated instrument 128 to position the object within and/or adjacent to distal end 132 of integrated instrument 128.

At step 408, process 400 includes moving the instrument actuation mechanism (e.g., instrument actuator 148) from the second position (e.g., second position 152) toward first position (e.g., first position 150) to cause distal end 132 of integrated instrument 128 to at least partially retract relative to tube 104 and transition to an at least partially collapsed state. For example, once the PEG wire is captured with distal end 132 of integrated instrument 128, the operator of medical device 100 may move instrument actuator 148 from second position 152 toward first position 150, such that integrated instrument 128 at least partially retracts relative to tube 104 (e.g., retracts toward distal end 108). At least partially retracting integrated instrument 128 relative to tube 104 may help to return distal end 132 of integrated instrument 128 to an at least partially collapsed state, which may help to secure the PEG wire. At step 410, process 400 may include removing medical device 100 from the patient's body. For example, the entirety of medical device 100 may be removed from the patient, which also removes the PEG wire from the patient. Any remaining steps of the procedure may be completed (e.g., for which medical device 100 is not used). The medical device 100 may be a single-use device that is then disposed.

Process 400 described above is provided merely as an example, and may include additional, fewer, different, or differently arranged steps than depicted in FIG. 4 and as described above. Furthermore, the steps of process 400 may be performed with one or more of the medical devices described herein (i.e., medical device 200 or medical device 300).

Based on the integration of integrated instrument 128 and instrument actuation mechanism 146 into medical device 100 (e.g., as opposed to conventional separate accessory devices with separate controls that are inserted into and fed through a working lumen of medical device 100), an assistant is no longer needed for operating medical device 100 (albeit a second operator located near abdomen of patient may still be utilized to place the PEG tube). A single operator of medical device 100 can actuate instrument actuator 148 of instrument actuation mechanism 146 to extend and retract integrated instrument 128 relative to tube 104 to transition between collapsed and expanded states and/or rotate medical device 100 to rotate integrated instrument 128. Elimination of the assistant for operating medical device 100 may be critical in certain service settings, such as the intensive care unit bedside setting, where there is limited space for two operators to be side by side, particularly near the head of the patient.

While the specific application of medical device 100 described herein is associated with a PEG procedure following a percutaneous tracheostomy, medical device 100 is not limited to this application. For example, medical device 100 may be used for any procedure in which an instrument that is typically a separate device or accessory is inserted into and delivered to a site within the patient's body via a working lumen of the medical device. As one specific example, medical device 100 may be used to perform a foreign body retrieval procedure. For example, a patient may have swallowed an object, such as, for example, a paper clip, and medical device 100 may have an integrated snare, basket, net, or other similar instrument, which may be used to retrieve the object.

FIGS. 2A-2D depict block diagrams of another exemplary medical device 200 for performing a medical procedure. Medical device 200 is similar to medical device 100, except for medical device 200 includes a sheath 202 separate from the lumen of tube 104 in which integrated instrument 128 is disposed. Sheath 202 may be a hollow, tubular member having a first proximal opening and a second distal opening. Based on the inclusion of sheath 202, medical device 200 also has a different instrument actuation mechanism 204 (e.g., replacing instrument actuation mechanism 146 of medical device 100) for controlling an extension and a retraction of sheath 202 and integrated instrument 128.

Actuation mechanism 204 may include a sheath actuator 206 that is disposed in a sheath track 208, and sheath actuator 206 may be coupled to a proximal end of sheath 202. Coupling may include a direct coupling or an indirect coupling (e.g., via one or more intermediate structures). Sheath actuator 206 may be movable between a first position 210 (FIGS. 2A and 2B) and a second position 212 (FIGS. 2C and 2D) within sheath track 208 to extend and retract sheath 202 with integrated instrument 128 disposed therein relative to tube 104. Actuation mechanism 204 may also include an instrument actuator 214 that is disposed in an instrument track 216, and instrument actuator 214 may be coupled to proximal end 130 of integrated instrument 128. Instrument actuator 214 may be movable between a third position 218 (FIGS. 2A, 2B, and 2C) and a fourth position 220 (FIG. 2D) within instrument track 216 to extend and retract at least distal end 132 of integrated instrument 128 relative to sheath 202 to transition distal end 132 between a collapsed state and an expanded state. Instrument track 216 may be a portion of sheath track 208 defined by sheath actuator 206 (e.g., instrument track 216 may be enclosed by sheath actuator 206).

FIG. 2A depicts a top view, and FIG. 2B depicts a side view of medical device 200 in a first configuration. In the first configuration of medical device 200, sheath actuator 206 may be in first position 210 within sheath track 208, and instrument actuator 214 may be in third position 218 within instrument track 216. Resultantly, in the first configuration of medical device 200, both sheath 202 and integrated instrument 128 are retracted relative to tube 104, and integrated instrument 128 is retracted relative to sheath 202, for example, in a collapsed state. Distal end 203 of sheath 202 may be positioned at distal end 108 of tube 104 (e.g., within articulation joint 109).

FIG. 2C depicts a top view of medical device 200 in a second configuration following operator movement of sheath actuator 206 from first position 210 to second position 212 within sheath track 208. In the second configuration, at least distal end 203 of sheath 202 in which integrated instrument 128 is disposed may be extended relative to tube 104 as a result of the operator movement. For example, sheath 202 may extend distally past tube 104 via distal opening 114 in cap 110. Based on instrument actuator 214 remaining in third position 218 within instrument track 216, distal end 132 of integrated instrument 128 may remain retracted relative to sheath 202 in a collapsed state when medical device 200 is in the second configuration.

FIG. 2D depicts a top view of the medical device 200 in a third configuration following operator movement of instrument actuator 214 from third position 218 to fourth position 220 within instrument track 216 (and while sheath actuator 206 remains in second position 212). In the third configuration, at least distal end 132 of integrated instrument 128 may be extended relative to sheath 202 and may be transitioned from a collapsed state to an expanded state. For example, distal end 132 of integrated instrument 128 may extend distally from distal end 203 of sheath 202 via a distal opening in the sheath 202.

Once an object of interest is positioned relative to distal end 132 of integrated instrument 128 in the expanded state (i.e., within distal end 132), the operator may move instrument actuator 214 from fourth position 220 toward third position 218. Movement of instrument actuator 214 from fourth position 220 toward third position 218 may at least partially retract distal end 132 of integrated instrument 128 relative to sheath 202 and transition distal end 132 of integrated instrument 128 from the expanded state to an at least partially collapsed state to secure the object of interest. In some examples, the operator may simultaneously or consecutively move sheath actuator 206 from second position 212 toward first position 210 to at least partially retract sheath 202 and integrated instrument 128 relative to tube 104 prior to removing an entirety of medical device 200 from the patient. Alternatively, the operator may instead remove the entirety of medical device 200 from the patient without partially retracting sheath 202 and integrated instrument 128 relative to tube 104. In some examples, medical device 200 may include one or more locking features to secure sheath actuator 206 and/or instrument actuator 214 in a given position (e.g., in one of first position 210 or second position 212 or in one of third position 218 or fourth position 220, respectively).

By including a dedicated sheath, such as sheath 202, in which integrated instrument 128 is disposed in medical device 200, the operating field may be enlarged to include the length of sheath 202, which may be up to a length of handle 102, as well as the length of integrated instrument 128 when extended distally from sheath 202. Additionally, after distal end 132 of integrated instrument 128 is at least partially retracted relative to sheath 202 and transitioned to the at least partially collapsed state, sheath 202 may help to enable a tighter and/or more secure closure of the object of interest by integrated instrument 128 within the collapsed state. In these aspects, sheath 202 may help to provide a more secure grip on the object of interest as medical device 200 is being removed from patient.

Medical device 200 may be used to perform a medical procedure using similar steps described above with reference to process 400 of FIG. 4. One or more additional steps may be included to account for differences in instrument actuation mechanism 204 of medical device 200, as described above.

FIGS. 3A-3C depict perspective views of a distal portion of a further exemplary medical device 300 for performing a medical procedure. Medical device 300 is similar to medical device 100 described in FIGS. 1A-1D except for the integrated instrument is a wire 302 that, based on the integration of wire 302 within medical device 300, forms a loop or snare that can be transitioned between a collapsed state and an expanded state using an instrument actuation mechanism, for example, instrument actuation mechanism 146. Although the term “wire” is used herein for wire 302, it will be appreciated that the disclosure also encompasses cables, threads, or other similar structures. For example, a distalmost end 304 of wire 302 may be affixed to distal-facing surface 112 of cap 110 at a fixation point 306. From fixation point 306, wire 302 may extend into tube 104 via an opening 308 in cap 110. In some examples, opening 308 may correspond to a wire lumen separate from the lumen to which distal opening 114 corresponds. In other examples, opening 308 may connect to the same lumen to which opening 114 corresponds. Wire 302 may continue to extend from tube 104 into handle 102 via distal opening 168 of connector 166 and exit via instrument connector port 172, where a proximalmost end of wire 302 (not shown) is to attached to driver 154 (FIGS. 1C and 1D). As previously described with reference to FIGS. 1A-1D, driver 154 may be connected to an interior portion of instrument actuator 148 by link 156. Accordingly, when instrument actuator 148 is moved by the operator between first position 150 and second position 152, driver 154 and wire 302 correspondingly move to extend and/or retract a portion of wire 302 via opening 308 to transition a loop or snare formed by wire 302 at the distal end of medical device 300 between collapsed and expanded states. In some examples, portions of wire 302 within tube 104 and/or handle 102 (e.g., portions of wire 302 that are more proximally positioned and do not extend from opening 308 when wire 302 is in a fully expanded state) may be enclosed within a compression cable.

In medical device 300, opening 308 via which wire 302 is extended may be a separate opening than the distal opening 114. Additionally, in some examples and as shown in FIGS. 3A-3C, fixation point 306 and opening 308 may be positioned equidistantly on either side of distal opening 114 and offset from distal opening 114, which may help to prevent wire 302 from blocking distal opening 114. Accordingly, other accessory devices, tools, or instruments separate from medical device 300 that may be utilized for the medical procedure may be inserted into working lumen access 180, through connector 166 via working lumen connector port 178, and into tube 104 via distal opening 168 of connector 166, for example, as discussed above with respect to FIG. 1C. The other accessory devices, tools, or instruments may then be extended through tube 104 for delivery to an intended site within the patient's body via distal opening 114 without being blocked by wire 302. Additionally, fluid supplied and/or suction applied via fluidics device 160 is not obstructed by wire 302. Further, wire 302 would not impair a visibility of imaging device 120 and/or illumination of illumination device(s) 122.

FIG. 3A depicts the distal portion of medical device 300 in a first configuration where the loop or snare formed by wire 302 is in a collapsed state, for example, based on instrument actuator 148 being in first position 150. For example, in the collapsed state, the portion of wire 302 extending between distal end 304 of wire 302 and opening 308 may at least partially contact distal-facing surface 112 of cap 110. FIG. 3B depicts the distal portion of the medical device 300 in a second configuration, where the loop or snare formed by wire 302 is in an expanded state, for example, based on instrument actuator 148 being moved from first position 150 to second position 152. For example, movement of instrument actuator 148 to second position 152 causes driver 154 to correspondingly move wire 302 distally such that a portion of wire 302 previously positioned within distal end 108 of tube 104 extends distally from opening 308 to transition wire 302 to an expanded state (e.g., forming a larger loop or snare). Once the operator positions an object of interest relative to wire 302 in the expanded state, the operator may then move instrument actuator 148 from second position 152 toward first position 150 to transition wire 302 back to an at least partially collapsed state (as in FIG. 3A) to help secure the object with wire 302. For example, in the partially collapsed state, wire 302 may secure the object such that the object at least partially contacts distal-facing surface 112 of cap 110.

FIG. 3C depicts an alternative configuration of distal-facing surface 112. In this alternative configuration, distal-facing surface 112 may include a longitudinal recess 310 positioned between fixation point 306 and opening 308. Recess 310 may be configured to receive at least a portion of an object 312 retrieved during a medical procedure, such as a PEG wire during a PEG. In some examples, recess 310 may be sized and/or shaped to correspond to the size and/or shape of object 312. Recess 310 may help to allow for a greater cross-sectional area of distal-facing surface 112 to contact object 312 to help to provide a more secure grip of object 312 as the medical device 300 is then removed from the patient.

As described above with reference to FIGS. 3A-3C, medical device 300 has one-sided actuation that is controlled via instrument actuation mechanism 146. With one-sided actuation, an entirety of a length of wire 302 needed to transition wire 302 from the collapsed to expanded state is extended from opening 308, which may result in a longer stroke length for instrument actuator 148. To reduce stroke length (e.g., by half), medical device 300 may be modified to have two-sided actuation. In such an alternative aspect, instead of distal end 304 of wire 302 being attached to distal-facing surface 112 at fixation point 306, distal-facing surface 112 may include another opening at the location of fixation point 306, and distal end 304 of wire 302 may extend into tube 104 via the other opening. For clarity, when describing the two-sided actuation alternative, distal end 304 of wire 302 is now referred to as a second end of wire 302 that is opposite a first end of wire 302 previously referred to as the proximalmost end of wire 302. The second end of wire 302 may continue to extend from tube 104 into handle 102 via distal opening 168 of connector 166 and exit via instrument connector port 172, where the second end of wire 302 is then attached to an instrument actuation mechanism.

In some examples, the second end of wire 302 may be attached to the same instrument actuation mechanism as the first end of wire 302. For example, both first end and second end of wire 302 may be attached to driver 154 of instrument actuation mechanism 146 (FIGS. 1C and 1D). Accordingly, the operator may move a single instrument actuator 148 to cause wire 302 to be extended distally from both opening 308 and other opening simultaneously to transition wire 302 to the expanded state with half the stroke length.

In other examples, handle 102 may include another instrument actuation mechanism similar to instrument actuation mechanism 146, where the first end of wire 302 may be attached to instrument actuation mechanism 146, and the second end of wire 302 may be attached to the other instrument actuation mechanism. The operator may actuate the two separate instrument actuation mechanisms to cause wire 302 to be extended from opening 308 and the other opening to transition wire 302 to the expanded state with half the stroke length. In some examples, the two separate instrument actuation mechanisms may be actuated simultaneously to cause a substantially equal amount of wire 302 to be extended from each of opening 308 and the other opening. In other examples, the two separate instrument actuation mechanisms may be actuated individually to selectively cause a different amount of wire 302 to be extended from each of opening 308 and the other opening (e.g., causing the loop formed from wire 302 to be expanded relative to position of opening from which a greater amount of wire 302 is extended to better approach the object, for example).

Medical device 300 may be used to perform a medical procedure using the same or similar steps described above with reference to process 400 of FIG. 4. In some examples, the object of interest captured at step 406 (e.g., the PEG wire) may be retained within recess 310 as part of at least one of steps 406, 408, and/or 410. Additionally, in examples where handle 102 includes the two separate instrument actuation mechanisms (e.g., instrument actuation mechanism 146 and other instrument actuation mechanism similar to instrument actuation mechanism 146), one and/or both of the instrument actuation mechanisms may be moved between first and second positions at steps 404 and 408.

While principles of this disclosure are described herein with the reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

We claim:

1. A medical device, comprising:
a tube having a proximal end and a distal end, and comprising a lumen extending from the proximal end to the distal end;
a cap at the distal end of the tube, the cap having a distal opening corresponding to the lumen;
an imaging system positioned on a distal-facing surface of the cap;
an instrument at least partially disposed in the lumen that is non-removable from the medical device;
an instrument actuation mechanism for controlling the instrument; and
a single handle connected to the proximal end of the tube and housing the instrument actuation mechanism,
wherein a proximal end of the instrument is affixed to the instrument actuation mechanism and the instrument actuation mechanism is configured to be moved between a first position and a second position on the single handle to control an extension and retraction of the instrument relative to the tube,
wherein a distal end of the instrument is retracted relative to the tube in a collapsed state when the instrument actuation mechanism is in the first position, and
wherein the distal end of the instrument is extended relative to the tube in an expanded state when the instrument actuation mechanism is in the second position.

2. The medical device of claim 1, wherein the distal end of the tube includes an articulation joint, and the distal end of the instrument is positioned within the articulation joint when the instrument actuation mechanism is in the first position.

3. The medical device of claim 2, wherein the single handle further houses an articulation actuation mechanism for controlling the articulation joint, the articulation joint is configured to bend in at least two directions away from a longitudinal axis of the tube.

4. The medical device of claim 1, wherein the instrument is extended relative to the tube via the distal opening, and the distal opening includes a clamshell shape to lock an orientation of the instrument relative to the medical device as the instrument is extended.

5. The medical device of claim 1, further comprising a connector disposed within the single handle, wherein the connector includes:
a plurality of connector ports, wherein at least one of the plurality of connector ports includes an instrument connector port configured to receive the instrument from the instrument actuation mechanism; and
a distal opening connecting the single handle to the proximal end of the tube, wherein the instrument received via the instrument connector port extends into the lumen of the tube via the distal opening.

6. The medical device of claim 1, wherein the instrument actuation mechanism includes an instrument actuator configured to be moved between the first position and the second position, a link, and a driver connected to the instrument actuator via the link to which the proximal end of the instrument is attached.

7. The medical device of claim 1, further comprising:
a sheath at least partially disposed within the lumen, wherein the instrument is disposed within the sheath.

8. The medical device of claim 7, wherein the instrument actuation mechanism comprises:
a sheath actuator that extends and retracts the sheath in which the instrument is disposed relative to the tube; and
an instrument actuator that extends and retracts the instrument relative to the sheath to transition the instrument between the collapsed state and the expanded state.

9. The medical device of claim 1, wherein:
the instrument is a wire,
a first end of the wire is attached to a fixation point on the distal-facing surface of the cap,
the wire extends from the fixation point through a second distal opening of the cap, different from the distal opening corresponding to the lumen, into the handle via the tube, and
a second end of the wire opposite the first end of the wire is affixed to the instrument actuation mechanism.

10. The medical device of claim 9, wherein:
when the instrument actuation mechanism is in the first position, a first portion of the wire from the fixation point to the second distal opening forms a loop in the collapsed state that at least partially contacts the distal-facing surface; and
when the instrument actuation mechanism is in the second position, a second portion of the wire is extended from the tube via the second opening to transition the loop to the expanded state.

11. The medical device of claim 9, wherein the distal-facing surface of the cap comprises a recess arranged between the fixation point and the second distal opening, the recess configured to receive and secure an object captured by the instrument.

12. The medical device of claim 1, wherein:
the instrument is a wire,
a first end of the wire extends through a second distal opening of the cap, different from the distal opening corresponding to the lumen, into the handle via the tube to attach to the instrument actuation mechanism, and
a second end of the wire extends through a third distal opening in the cap, different from the distal opening corresponding to the lumen and the second distal opening, into the handle via the tube to attach to the instrument actuation mechanism.

13. The medical device of claim 12, wherein:
when the instrument actuation mechanism is in the first position, a first portion of the wire from the second distal opening to the third distal opening forms a loop in the collapsed state that at least partially contacts the distal-facing surface; and
when the instrument actuation mechanism is in the second position, a second portion of the wire is extended from the tube via the second opening and a third portion of the wire is extended from the tube via the third opening to transition the loop to the expanded state.

14. The medical device of claim 1, wherein the single handle further comprises a fluidics device having a proximal end connectable to one or more of a fluid supply source and a vacuum source and a distal end connected to the proximal end of the tube, the fluidics device configured to one or more of supply fluid or apply suction to a patient's body via the distal opening.

15. The medical device of claim 1, wherein the imaging system includes an imaging device positioned within an imaging port on the distal-facing surface of the cap, and an illumination device positioned within an illumination port on the distal-facing surface of the cap.

16. A medical device, comprising:
a tube having a proximal end and a distal end, and comprising a lumen extending from the proximal end to the distal end;
a cap at the distal end of the tube, the cap having a distal opening corresponding to the lumen;
a sheath at least partially disposed within the lumen;
an instrument disposed within the sheath, wherein the sheath and the instrument are non-removable from the medical device;
an instrument actuation mechanism comprising a sheath actuator and an instrument actuator; and
a single handle connected to the proximal end of the tube and housing the instrument actuation mechanism,
wherein a proximal end of the sheath is affixed to the sheath actuator and the sheath actuator is configured to move between a first position and a second position on the single handle to control an extension and retraction of the sheath in which the instrument is disposed relative to the tube via the distal opening, and
wherein a proximal end of the instrument is affixed to the instrument actuator and the instrument actuator is configured to move between a third position and a fourth position on the single handle to control an extension and retraction of a distal end of the instrument relative to the sheath to transition the distal end of the instrument between a collapsed state and an expanded state.

17. The medical device of claim 16, wherein the distal end of the tube includes an articulation joint, and a distal end of the sheath, in which the distal end of the instrument is disposed, is positioned within the articulation joint when the sheath actuator is in the first position.

18. The medical device of claim 16, wherein the instrument is a snare and the medical device is configured to be used for a percutaneous endoscopic gastrostomy (PEG).

19. A medical device, comprising:
a tube having a proximal end and a distal end, and comprising a lumen extending from the proximal end to the distal end;
a cap at the distal end of the tube, the cap having a distal opening corresponding to the lumen;
a handle connected to the proximal end of the tube and housing an actuation mechanism configured to be moved between a first position and a second position on the handle; and
a wire having a first end and a second end, wherein the wire is at least partially disposed in the lumen, the first end of the wire is attached to a fixation point on a distal-facing surface of the cap, the wire extends from the fixation point through a second distal opening of the cap, different from the distal opening corresponding to the lumen, into the handle via the tube, and a second end of the wire opposite the first end of the wire is affixed to the actuation mechanism,
wherein, when the actuation mechanism is in the first position, a first portion of the wire from the fixation point to the second distal opening forms a loop in a collapsed state that at least partially contacts the distal-facing surface, and
wherein, when the actuation mechanism is in the second position, a second portion of the wire is extended from the tube via the second opening to transition the loop to an expanded state.

20. The medical device of claim 19, wherein the distal-facing surface of the cap comprises a recess arranged between the fixation point and the second distal opening, the recess configured to receive at least a portion of an object captured by the wire.
